(19) United States
(12) Patent Application Publication
(10) Pub. No.: US 2024/0255488 A1
(43) Pub. Date: Aug. 1, 2024

(54) OIL STATE DETECTION APPARATUS

(71) Applicant: NIPPON PILLAR PACKING CO., LTD., Osaka (JP)

(72) Inventors: Akira NAKATSU, Osaka-shi (JP); Kentaro WATANABE, Osaka-shi (JP); Yusuke NATSUHARA, Osaka-shi (JP)

(73) Assignee: NIPPON PILLAR PACKING CO., LTD., Osaka (JP)

(21) Appl. No.: 18/427,678

(22) Filed: Jan. 30, 2024

(30) Foreign Application Priority Data
Jan. 30, 2023 (JP) 2023-011659

(51) Int. Cl. G01N 33/28

(52) U.S. Cl. CPC G01N 33/2888; G01N 33/2835

(57) ABSTRACT

To simplify the configuration of an oil state detection apparatus. The oil state detection apparatus 100 includes an oscillation circuit having a coil 1 and a capacitor 2 and a detection device configured to detect the oscillatory frequency of the oscillation circuit and having an oscillator 3 and a PLL circuit 4. One of the coil 1 or the capacitor 2 is immersed in oil OL. The oscillation circuit and the detection device are arranged on the same circuit board 20.

### BACKGROUND

The present disclosure relates to an oil state detection apparatus that detects the amount of degradation substance contained in oil.

Conventionally, there has been known a method for determining degradation of oil used as lubricant in, e.g., a vehicle or a construction machine.

An oil check sensor of Japanese Unexamined Patent Publication No. 2000-321248 includes a detection coil head and an oscillation tuned circuit. The detection coil head adsorbs metal powder in engine oil. The oscillation tuned circuit generates a detection signal having an amplitude changing in accordance with the inductance value of the detection coil head changing in accordance with the amount of adsorbed metal powder.

### SUMMARY

In Japanese Unexamined Patent Publication No. 2000-321248, the detection coil head and the oscillation tuned circuit are separately provided, and for this reason, need to be separately produced and be electrically connected to each other. This leads to a complicated sensor configuration and an increase in a cost due to an increase in the number of assembly steps.

The present disclosure is intended to provide an oil state detection apparatus having a simple configuration.

In order to accomplish the above-described object, an oil state detection apparatus according to one embodiment of the present disclosure includes a first oscillation circuit including a first coil and a first capacitor, and a first detection device that detects the oscillatory frequency of the first oscillation circuit. One of the first coil or the first capacitor is immersed in oil, and the first oscillation circuit and the first detection device are arranged on the same circuit board.

According to this configuration, since the first oscillation circuit and the first detection device are arranged on the same circuit board, the configuration of the oil state detection apparatus can be simplified without the need for separately providing the oscillation circuit and the detection device.

According to the present disclosure, the configuration of the oil state detection apparatus can be simplified.

### BRIEF DESCRIPTION OF THE DRAWINGS

FIG. 1 is a block diagram showing an oil state detection apparatus according to a first embodiment.

FIG. 2 is a graph showing a relationship between a tuning voltage and the amount of magnetic substance contained in oil according to the first embodiment.

FIGS. 3A and 3B are sectional views showing the configuration of the oil state detection apparatus according to the first embodiment.

FIG. 4 is a longitudinal sectional view showing the configuration of an oil state detection apparatus according to a second embodiment.

FIG. 5 is a graph showing the operation temperature of a detection device according to the second embodiment.

FIGS. 6A and 6B are sectional views showing the configuration of an oil state detection apparatus according to a modification of the second embodiment.

FIGS. 7A and 7B are views showing the configuration of a circuit board according to a third embodiment.

FIG. 8 is a view showing the configuration of a circuit board according to a first modification of the third embodiment.

FIG. 9 is a view showing the configuration of a circuit board according to a second modification of the third embodiment.

FIGS. 10A and 10B are views showing the configuration of a circuit board according to a third modification of the third embodiment.

FIG. 11 is a view showing the configuration of the vicinity of a tip end portion of an oil state detection apparatus according to another example of the first embodiment.

### DETAILED DESCRIPTION

Hereinafter, embodiments of the present disclosure will be described in detail with reference to the drawings. The following description of the preferred embodiments is merely an example in nature, and is not intended to limit the scope, application, and use of the present disclosure.

### First Embodiment

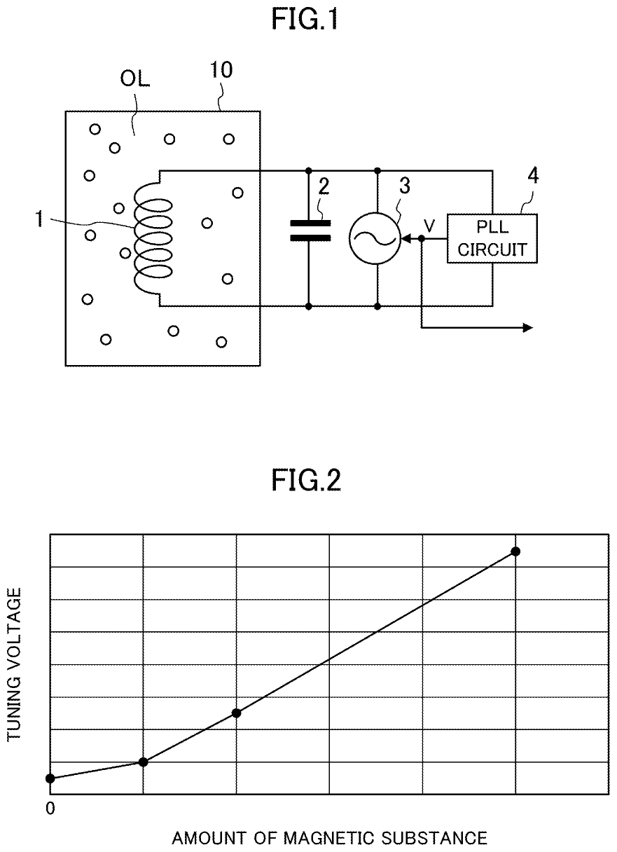

FIG. 1 is a block diagram showing an oil state detection apparatus according to a first embodiment. As shown in FIG. 1, this oil state detection apparatus includes a coil 1 (first coil), a capacitor 2 (first capacitor), an oscillator (e.g., voltage controlled oscillator (VCO)) 3, and a phase locked loop (PLL) circuit 4. Note that the oscillator 3 and the PLL circuit 4 are equivalent to a first detection device.

As shown in FIG. 1, the coil 1 is arranged inside a case 10 filled with oil OL (lubricant). The case 10 is, for example, an oil pan or an oil tank, and in the case 10, the oil OL circulates in a vehicle or a construction machine. Alternatively, the case 10 is a gearbox or a gearbox casing, and in the case 10, the oil OL is accumulated in a vehicle or a construction machine. The coil 1 is immersed in the oil OL inside the case 10. Thus, the inductance of the coil 1 changes in accordance with the amount of magnetic substance contained in the oil OL.

Here, the oscillatory frequency F of an oscillation circuit (equivalent to a first oscillation circuit, and hereinafter, the oscillation circuit including the coil 1 and the capacitor 2 may be simply referred to as an “oscillation circuit”) including the coil 1 and the capacitor 2 is represented by the following equation:

### [Equation 1]

where L is the inductance of the coil 1 and C is the capacity of the capacitor 2.

The PLL circuit 4 inputs a tuning voltage V to the oscillator 3 such that an output frequency output by the oscillator 3 satisfies Equation (1).

FIG. 2 is a graph showing a relationship between the tuning voltage V and the amount of magnetic substance contained in the oil. In FIG. 2, the vertical axis indicates the tuning voltage V, and the horizontal axis indicates the amount of magnetic substance.

As shown in FIG. 2, as the amount of the magnetic substance increases, the voltage value of the tuning voltage V increases. This is because the inductance L of the coil 1 changes (increases or decreases) with an increase in the amount of magnetic substance contained in the oil OL. Thus, the PLL circuit 4 increases the tuning voltage V in order to decrease the output frequency of the oscillator 3 such that the output frequency of the oscillator 3 traces the oscillatory frequency of the oscillation circuit (see Equation (1)). Thus, by detecting the voltage value of the tuning voltage V, i.e., the output frequency of the oscillator 3, the amount of magnetic substance (amount of degradation substance) contained in the oil OL can be detected.

Further, as shown in FIG. 2, the present oil state detection apparatus includes the coil 1, the capacitor 2, the oscillator 3, and the PLL circuit 4. Thus, for example, the inductance value of the coil 1 can be decreased by increasing the oscillatory frequency of the oscillation circuit (coil 1 and capacitor 2). This allows size reduction in the oil state detection apparatus. Note that the oscillator 3 normally includes a semiconductor element, and therefore, the size thereof does not change in accordance with a change in the output frequency.

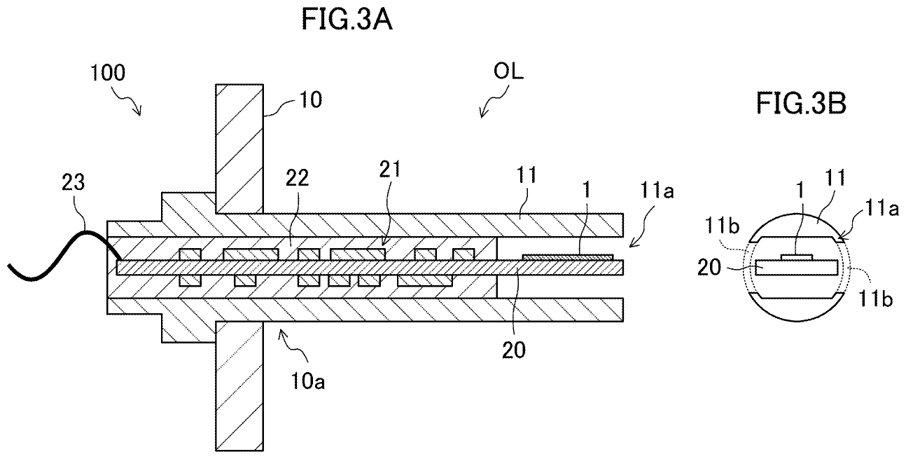

FIGS. 3A and 3B are sectional views showing the configuration of the oil state detection apparatus according to the first embodiment. Specifically, FIG. 3A is a longitudinal sectional view of an oil state detection apparatus 100, and FIG. 3B is a cross-sectional view of the oil state detection apparatus 100. Note that in FIG. 3A, the right side of the case 10 as viewed in the figure is the inside of the case 10 and the left side of the case 10 as viewed in the figure is the outside of the case 10.

As shown in FIG. 3A, the present oil state detection apparatus includes a housing 11 and a circuit board 20.

The housing 11 is a case covering the circuit board 20 etc. The housing 11 is, for example, a case made of metal or resin. The housing 11 is arranged apart from the coil 1, and therefore, functions as a counter electrode for the coil 1. The circuit board 20 is arranged inside the housing 11. Moreover, the housing 11 is inserted into a through-hole 10a formed in the case 10, and a tip end portion (right end portion as viewed in the figure) thereof is immersed in the oil OL.

The circuit board 20 is, for example, a circuit board made of fluorine compound resin. The coil 1 and an electronic circuit element 21 are arranged on the circuit board 20. The electronic circuit element 21 includes the capacitor 2, the oscillator 3, the PLL circuit 4, etc. Specifically, the coil 1 is arranged on the tip end side (right side as viewed in the figure) of the circuit board 20, and is immersed in the oil OL. Moreover, on the circuit board 20, the electronic circuit element 21 is formed on the base end side (left side as viewed in the figure) with respect to the coil 1. The electronic circuit element 21 is covered with sealing resin 22 such as epoxy or urethane. The electronic circuit element 21 is connected to an external apparatus (not shown) via a cable 23.

The coil 1 and the circuit board 20 are arranged in parallel with the longitudinal direction of the housing 11. That is, the coil 1 is arranged along the longitudinal direction of the housing 11. Thus, as compared to a case where the coil 1 is arranged perpendicularly to the longitudinal direction of the housing 11, the area of the coil 1 can be greater and the resolution of the oil state detection apparatus can be improved.

As shown in FIG. 3B, the housing 11 is formed in a substantially cylindrical shape. The tip end portion of the housing 11 is formed with an opening 11a. Moreover, both right and left surfaces of the tip end portion of the housing 11 are formed with cutouts 11b. The oil OL flows into the tip end portion of the housing 11 through the opening 11a and the cutouts 11b, and the coil 1 is immersed in the oil OL. Note that the cutouts 11b may be formed in both upper and lower surfaces of the tip end portion of the housing 11.

With the above-described configuration, the oil state detection apparatus according to the present embodiment includes the oscillation circuit (first oscillation circuit) including the coil 1 (first coil) and the capacitor 2 (first capacitor), and the detection device (first detection device) configured to detect the oscillatory frequency of the oscillation circuit and including the oscillator 3 and the PLL circuit 4. The coil 1 is immersed in the oil OL. The oscillation circuit and the detection device are arranged on the same circuit board 20. Since the oscillation circuit and the detection device are arranged on the same circuit board 20, the configuration of the oil state detection apparatus can be simplified without the need for separately providing the oscillation circuit and the detection device.

### Second Embodiment

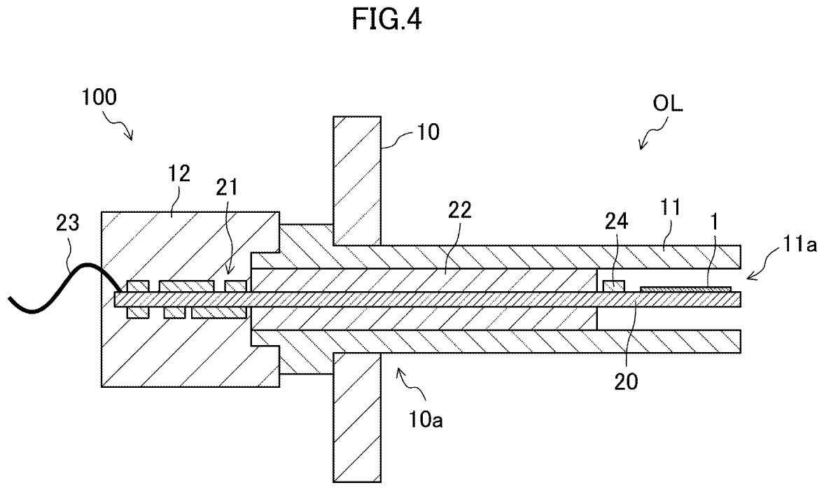

FIG. 4 is a longitudinal sectional view showing the configuration of an oil state detection apparatus according to a second embodiment. The second embodiment is different from the first embodiment in the arrangement of an electronic circuit element 21.

Specifically, the electronic circuit element 21 is arranged inside the housing 11 in the first embodiment, but a housing 12 is provided at a base end portion (left end portion as viewed in the figure) of a housing 11 in the second embodiment. The electronic circuit element 21 is arranged inside the housing 12.

The housing 12 (second housing) is, for example, a case made of metal or resin. The housing 12 is arranged, outside the case 10, apart from the case 10.

A temperature sensor 24 is arranged on the tip end side (right side as viewed in the figure) of a circuit board 20. The temperature sensor 24 is immersed in oil OL, and detects the temperature of the oil OL.

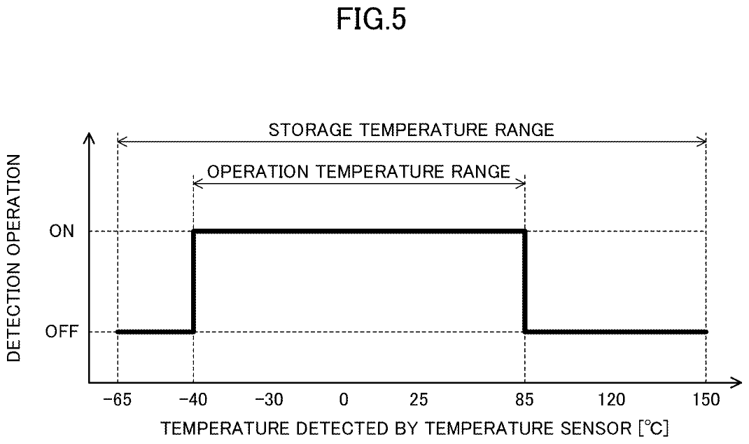

FIG. 5 is a graph showing the operation temperature of a detection device according to the second embodiment. As shown in FIG. 5, the detection device operates at −40° C. to 85° C. within an operation guarantee temperature range, and stops outside the operation guarantee temperature range. Note that the detection device operates/stops according to the temperature detected by the temperature sensor 24.

Here, the oil OL is lubricant circulating inside, e.g., a vehicle, and therefore, tends to be at a high temperature when, e.g., the vehicle is driven. For this reason, in a case where the detection device is arranged inside the housing 11, the oil OL inside the case 10 and the detection device are close to each other, and therefore, the detection device is susceptible to a change in the temperature of the oil OL. Thus, there is a probability that the detection device cannot operate within the operation guarantee temperature range.

For this reason, in the present embodiment, the detection device (electronic circuit element 21) is arranged, outside the case 10, inside the housing 12 arranged apart from the case 10. With this configuration, the influence of the change in the temperature of the oil OL on the detection device can be reduced, and therefore, the detection device can operate within the operation guarantee temperature range.

Note that in a case where the detection device can operate within the operation guarantee temperature range even if the electronic circuit element 21 is arranged inside the housing 11, such an electronic circuit element may be arranged inside the housing 11. That is, an electric circuit element, which has a heat resistance performance of a predetermined temperature or less, of the detection device is only required to be arranged inside the housing 12.

### Modification

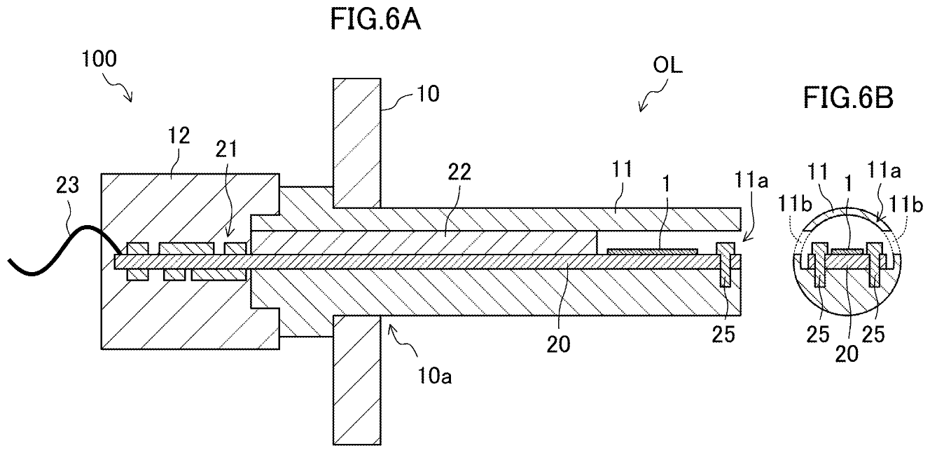

FIGS. 6A and 6B are sectional views showing the configuration of an oil state detection apparatus according to a modification of the second embodiment. Specifically, FIG. 6A is a longitudinal sectional view of the oil state detection apparatus 100, and FIG. 6B is a cross-sectional view of the oil state detection apparatus 100.

As shown in FIGS. 6A and 6B, the circuit board 20 is mounted on a lower portion of the housing 11. The circuit board 20 is fixed to the housing 11 via a fixing portion 25. The fixing portion 25 is, for example, a screw or a bolt.

With the configuration of FIGS. 6A and 6B, the circuit board 20 is fixed to the housing 11 via the fixing portion 25 so that the mechanical strength of the oil state detection apparatus 100 can be improved. Particularly, in a case where vibration or impact is easily applied to the oil state detection apparatus 100, the configuration of FIGS. 6A and 6B is useful.

### Third Embodiment

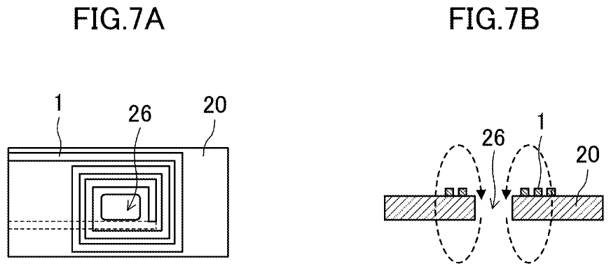

FIGS. 7A and 7B are views showing the configuration of a circuit board according to a third embodiment. Specifically, FIG. 7A is an enlarged plan view of a portion of the circuit board 20 on which a coil 1 is arranged, and FIG. 7B is a sectional view of the portion of the circuit board 20 on which the coil 1 is arranged.

As shown in FIGS. 7A and 7B, the circuit board 20 is formed with a through-hole 26 at a position corresponding to the center of the coil 1. Thus, oil OL flows into the through-hole 26. Accordingly, a magnetic field is intensified at the center of the coil 1, and therefore, micrometal contained in the oil OL flowing into the through-hole 26 can be detected.

### First Modification

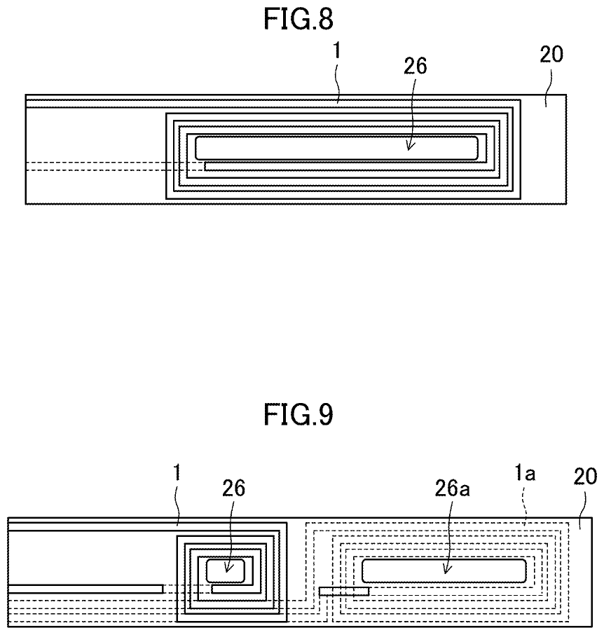

FIG. 8 is a view showing the configuration of a circuit board according to a first modification of the third embodiment. Specifically, FIG. 8 is an enlarged plan view of the portion of the circuit board 20 on which the coil 1 is arranged. As shown in FIG. 8, the width of the coil 1 in the right-left direction as viewed in the figure may be increased.

### Second Modification

FIG. 9 is a view showing the configuration of a circuit board according to a second modification of the third embodiment. Specifically, FIG. 9 is an enlarged plan view of a portion of the circuit board 20 on which the coils 1, 1a are arranged. As shown in FIG. 9, the coil 1 is arranged on the upper surface of the circuit board 20, and the coil 1a is arranged on the back surface of the circuit board 20. Note that the circuit board 20 is formed with a through-hole 26a at a position corresponding to the center of the coil 1a.

In FIG. 9, the coils 1, 1a are immersed in the oil OL. Although not shown in the figure, a capacitor (second capacitor) corresponding to the coil 1a (second coil) and an oscillator and a PLL circuit (second detection device) are arranged on the circuit board 20 in FIG. 9. The coil 1a and the capacitor corresponding to the coil 1a are equivalent to a second oscillation circuit.

As shown in FIG. 9, the area of the coil 1 is narrower than the area of the coil 1a. That is, the coils 1, 1a are different from each other in area. With this configuration, the oil state detection apparatus can detect substances with different frequency characteristics.

### Third Modification

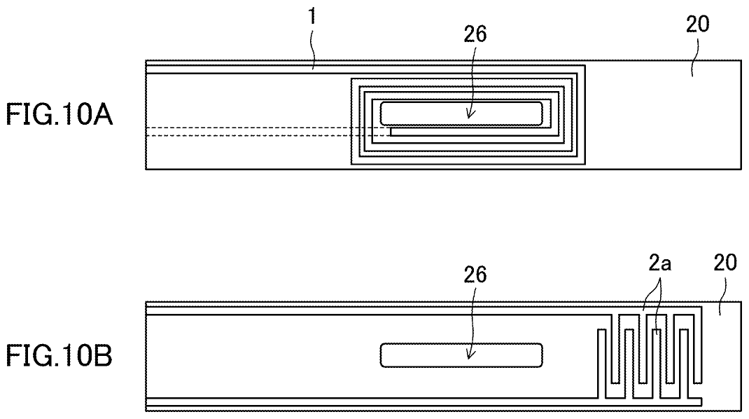

FIGS. 10A and 10B are views showing the configuration of a circuit board according to a third modification of the third embodiment. Specifically, FIGS. 10A and 10B are enlarged views of a portion of the circuit board 20 on which the coil 1 and a capacitor 2a (third capacitor) are arranged, FIG. 10A being a top view of the circuit board 20 and FIG. 10B being a bottom view of the circuit board 20. As shown in FIGS. 10A and 10B, the coil 1 is arranged on the upper surface of the circuit board 20, and the capacitor 2a is arranged on the lower surface of the circuit board 20. In FIGS. 10A and 10B, the coil 1 and the capacitor 2a are immersed in the oil OL.

As shown in FIGS. 10A and 10B, the capacitor 2a includes two comb-type electrodes. The capacitor 2a detects the resistance, capacity, and oscillatory frequency of the oil OL so that the detection value of the oscillation circuit including the coil 1 can be corrected. Thus, the amount of magnetic substance (amount of degradation substance) can be more accurately detected.

### Other Embodiments

Note that the oscillatory frequency of each oscillation circuit is detected by the detection device (oil state detection apparatus) including the oscillator and the PLL circuit in each of the above-described embodiments and modifications, but the configuration of the detection device may be a configuration other than the configuration including the oscillator and the PLL circuit. The detection device may have an arbitrary configuration as long as the detection device can detect the oscillatory frequency of the oscillation circuit.

The detection device includes the PLL circuit in each of the above-described embodiments and modifications, but the present disclosure is not limited thereto. For example, instead of the PLL circuit 4, the detection device may include an arithmetic circuit that analyzes an input frequency or an analysis circuit that decreases an input frequency and analyzes the frequency.

In each of the above-described embodiments and modifications, the capacitor 2 may be immersed in the oil OL instead of the coil 1. With this configuration, in each modification of the first embodiment, a corresponding effect can be obtained while the amount of dielectric substance (degradation substance) contained in the oil OL is detected.

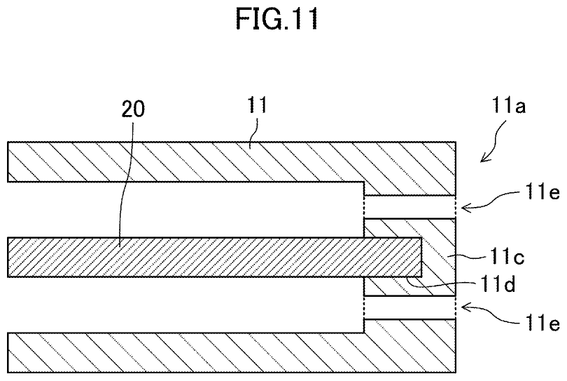

In each of the above-described embodiments and modifications, as shown in FIG. 11, a lid 11c may be provided to cover the opening 11a of the housing 11 and be formed with a slit 11d, and the tip end of the circuit board 20 may be inserted into the slit 11d. With this configuration, the mechanical strength of the oil state detection apparatus can be improved. Note that in FIG. 11, the lid 11c may be provided with a through-hole 11e and the oil OL may flow into the housing 11 through the through-hole 11e.

The oil state detection apparatus of the present disclosure is useful because the configuration thereof is simple.

What is claimed is:

1. An oil state detection apparatus comprising:
a first oscillation circuit including a first coil and a first capacitor; and
a first detection device that detects an oscillatory frequency of the first oscillation circuit,
wherein one of the first coil or the first capacitor is immersed in oil, and
the first oscillation circuit and the first detection device are arranged on an identical circuit board.

2. The oil state detection apparatus of claim 1, further comprising:
a first housing and a second housing that cover the circuit board,
wherein a tip end portion of the first housing is inserted into a case filled with the oil,
the second housing is arranged, at a base end portion of the first housing, apart from the case, and
an electric circuit element, which has a heat resistance performance of a predetermined temperature or less, of the first detection device is arranged inside the second housing.

3. The oil state detection apparatus of claim 1, further comprising:
a first housing that covers the circuit board; and
a fixing portion that fixes the circuit board to the first housing.

4. The oil state detection apparatus of claim 1, wherein
the circuit board is formed with a through-hole at a position corresponding to a center of the first coil.

5. The oil state detection apparatus of claim 1, further comprising:
a second oscillation circuit including a second coil having an area different from that of the first coil and a second capacitor; and
a second detection device that detects an oscillatory frequency of the second oscillation circuit,
wherein one of the second coil or the second capacitor is immersed in the oil,
the first oscillation circuit, the first detection device, the second oscillation circuit, and the second detection device are arranged on the identical circuit board, and
the first coil and the second coil are different from each other in the area.

6. The oil state detection apparatus of claim 1, further comprising:
a third capacitor immersed in the oil,
wherein the first oscillation circuit, the first detection device, and the third capacitor are arranged on the identical circuit board.

7. The oil state detection apparatus of claim 1, further comprising:
a temperature sensor immersed in the oil,
wherein according to a temperature detected by the temperature sensor, the first detection device operates within an operation guarantee temperature range, and stops outside the operation guarantee temperature range.
